(19) United States
(12) Patent Application Publication
(10) Pub. No.: US 2025/0166751 A1
(43) Pub. Date: May 22, 2025

(54) SYSTEMS AND METHODS FOR PATIENT RECORD MATCHING

(71) Applicant: Express Scripts Strategic Development, Inc., St. Louis, MO (US)

(72) Inventors: Narasimha Prasad Bingi, Parsippany, NJ (US); Sharmistha Vyas, Ridgewood, NJ (US); Roshan Kumar, Lake Hiawatha, NJ (US); Madhumitha Raghunathan, Parsippany, NJ (US)

(21) Appl. No.: 18/513,208

(22) Filed: Nov. 17, 2023

(51) Int. Cl. G16H 10/60; G16H 50/70

(52) U.S. Cl. CPC G16H 10/60; G16H 50/70

(57) ABSTRACT

A system and method may monitor a stream of messages from plural different sources of medical records to identify a new or updated record of the medical records, identifying one or more suspect records from a system of record (SOR) database. The one or more suspect records are identified as being potential matches to the new or updated record that is identified. The system and method can determine that the one or more suspect records match the new or updated record, and can obtain a person entity profile related to the new or updated record from a person entity database. The system and method can merge the new or updated record with the one or more suspect records or splitting the new or updated record from the one or more suspect records based on the person entity profile that is obtained.

### BACKGROUND

Health care is provided to patients using health care records that are associated with patients throughout their lives. Information can be included in these records to try and associate each record with a different patient. In the United States, however, there is no unique identifier assigned to each patient to ensure that each health care record is correctly associated with the same patient. Each health care provider, processor, and payor make individual decisions about for whom each service is being performed, and whether the patient receiving the service is the same as another patient who received another service on a different day. With millions of providers; thousands of insurers; patients constantly moving between providers and insurers; and a constant stream of demographic volatility and typographical errors, it can be very difficult to confidently decide who is who and which record represents the health care provided to which patient.

Along with variation of data across these multiple system sources, patient information itself may undergo variations throughout the lifetime of a patient. These variations in the patient information itself may arise due to changes in patient names, changes in the ways patients are contacted (e.g., contact mechanism changes), variations in insurance associations (e.g., patients change insurance providers), etc. Additionally, typographical errors may introduce variations in patient information.

Patient-matching processes attempt to determine whether two or more patient health care records are associated with the same patient. For example, these processes attempt to determine whether patient records from the same or different health care provider, processor, and/or payor represent health care received by the same person. These processes try to avoid overmatching the records, which occurs when multiple records are determined to belong to (e.g., are associated with) the same person when the records belong to different people. These processes also try to avoid undermatching, which occurs when multiple records are determined to belong to two different people when the records belong to the same person.

Both overmatching and undermatching can have significant consequences to health care decision-making. Overmatches can result in automated clinical decision-making using records that do not actually belong to the patient. Undermatches can result in automated clinical decision-making being performed without full view of the patient's records. Either overmatching or undermatching can pose serious risks to the health of patients. For example, contraindicated medications and/or treatments may be administered to patients due to overmatching or undermatching patient records. Many health care organizations err on the side of undermatches because overmatches may result also in inappropriate disclosures. For example, an overmatch could result in private information in a patient record of one patient being exposed (without permission) to another patient. Consequently, currently known systems provide users with a dilemma of risking undermatching records (which causes clinical decision-making to miss relevant information, with potentially deadly consequences) or overmatching records (which causes clinical decision-making to be made on the basis of incorrect information, as well as inappropriately exposing private health information). An improved system is needed that avoids both undesirable outcomes of this dilemma. That is, a system is needed that correctly matches patient records to reduce clinical risks and avoid risking inappropriate exposure of private health information.

### BRIEF DESCRIPTION

In one example, a method may include monitoring a stream of messages from plural different sources of medical records to identify a new or updated record of the medical records, identifying one or more suspect records from a system of record (SOR) database, the one or more suspect records identified as being potential matches to the new or updated record that is identified, determining that the one or more suspect records match the new or updated record, obtaining a person entity profile related to the new or updated record from a person entity database, and merging the new or updated record with the one or more suspect records or splitting the new or updated record from the one or more suspect records based on the person entity profile that is obtained.

In another example, a record matching system may include one or more data sync processors configured to monitor a stream of messages from plural different sources of medical records to identify a new or updated record of the medical records. The system also can include a person matching engine configured to identify one or more suspect records from an SOR database. The one or more suspect records are identified as being potential matches to the new or updated record that is identified. The person matching engine also can determine that the one or more suspect records match the new or updated record, obtain a person entity profile related to the new or updated record from a person entity database, and merge the new or updated record with the one or more suspect records or splitting the new or updated record from the one or more suspect records based on the person entity profile that is obtained.

In another example, a method may include monitoring a stream of messages from plural different sources of medical records to identify a new or updated record of the medical records, and identifying one or more suspect records from an SOR database. The one or more suspect records are identified as being potential matches to the new or updated record that is identified. The method also can include determining that the one or more suspect records match the new or updated record, and obtaining a person entity profile related to the new or updated record from a person entity database. The person entity profile can include source identifiers uniquely identifying which of the sources generated the medical records associated with each of several different persons. The method may include one or more of (a) merging the new or updated record with the one or more suspect records or (b) splitting the new or updated record from the one or more suspect records. Merging the new or updated record with the one or more suspect records occurs responsive to the source identifiers in the new or updated record and the one or more suspect records being associated with two or more of the different persons in the person entity database. Splitting the new or updated record from the one or more suspect records occurs responsive to the source identifiers in the new or updated record and the one or more suspect records being associated with a single person of the different persons in the person entity database along with one or more other source identifiers.

### BRIEF DESCRIPTION OF THE DRAWINGS

FIG. 1 is a functional block diagram of an example system including a high-volume pharmacy.

FIG. 2 is a functional block diagram of an example pharmacy fulfillment device, which may be deployed within the system of FIG. 1.

FIG. 3 is a functional block diagram of an example order processing device, which may be deployed within the system of FIG. 1.

FIG. 4 illustrates one example of a patient record matching system.

FIG. 5 illustrates a flowchart of one example of a method for determining whether records match each other.

FIG. 6 illustrates one example of a flowchart for a method of examining a new/updated record (e.g., a primary record) and one or more suspect records to determine whether these records match the same person or different persons.

FIG. 7 shows a block diagram of a computer system within which a set of instructions may be executed causing the machine to perform any one or more than one methods, processes, operations, or methodologies discussed herein.

FIG. 8 illustrates a functional block diagram of an example neural network that can be used by the record manager device for matching records with the same person.

In the drawings, reference numbers may be reused to identify similar and/or identical elements.

### DETAILED DESCRIPTION

Example systems and methods for matching patient records are described herein. These record matching systems and methods can perform efficient person entity resolution using intelligent patient matching algorithms over distributed computing. The systems and methods may employ an event streaming application for identifying changes to patient records and an intelligent matching rule engine for determining whether two or more patient records belong to or are associated with the same patient. The systems and methods can operate in real time or near real time (e.g., close to real time with unintentionally processing delays) to identify record changes as the changes occur, instead of performing a batch analysis (e.g., collecting a group of several records before examining for changes).

The event streaming application can perform entity resolution in near real-time while handling large data volumes (e.g., in the range of one billion source records and approximately two hundred fifty million records over approximately ten years of historical records). The event streaming application can rely on distributed computing and can be highly scalable by adding more worker nodes to the system to handle higher data volumes and/or improve performance throughputs. The intelligent matching rule engine can allow for independent rule authoring, testing, building, and deploying for matching rule consumption at runtime. The matching algorithm can operate within a deterministic system by applying fuzzy matching and pattern recognition techniques. To achieve high matching accuracy, entity relationships information, name pattern matching algorithms, entity locational patterns, and supervised learning from data stewards may be used.

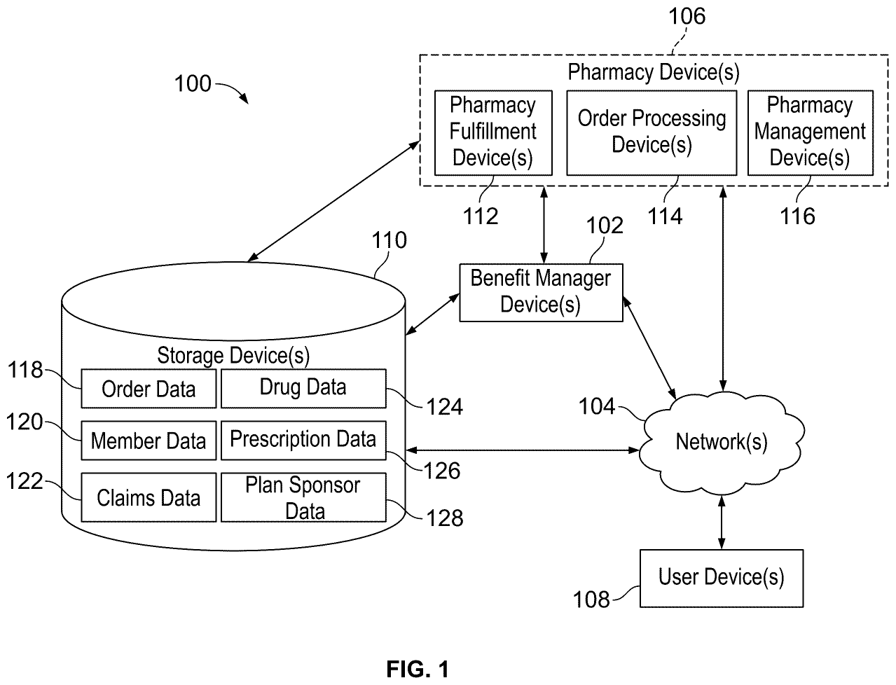

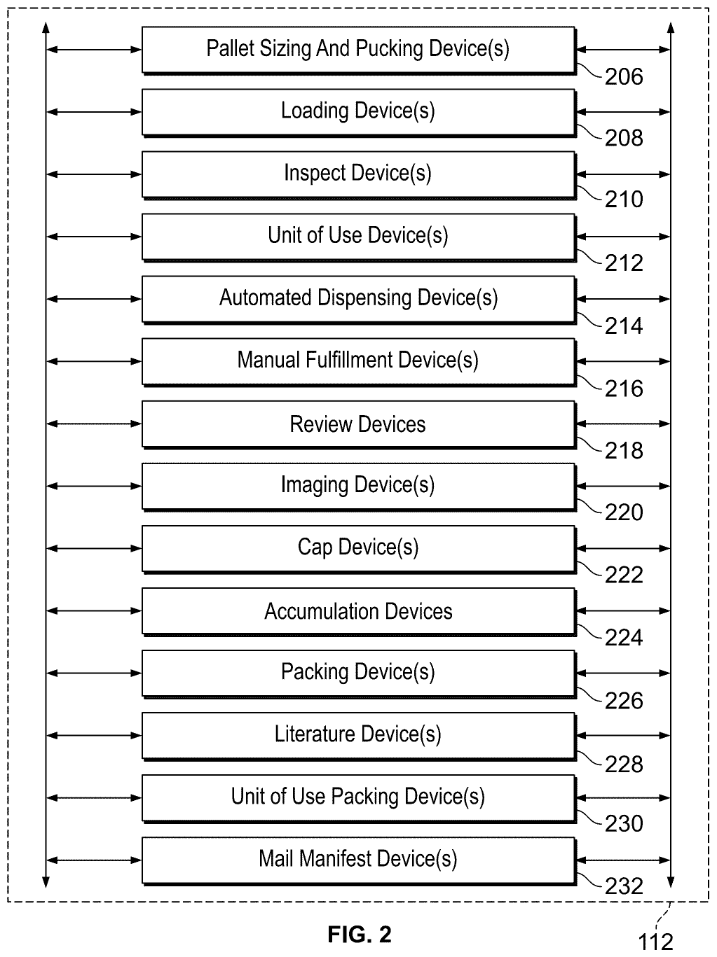

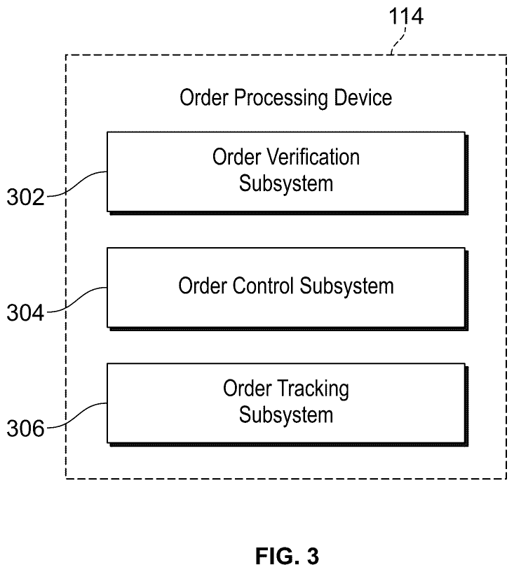

The inventive systems and methods are described herein in connection with high-volume pharmacy systems (e.g., FIG. 1), pharmacy fulfillment devices (e.g., FIG. 2), and pharmacy order processing devices (e.g., FIG. 3) to demonstrate various end uses for the patient records that are matched to the same patient (or identified as not matched to the same patient). Identifying which records match and do not match to the same patient can be useful in fulfilling high-volume pharmacy orders, filling other pharmacy orders, and the like, to ensure that the correct medication is provided to the correct person or patient. Not all embodiments of the inventive subject matter, however, are limited to filling pharmacy orders unless explicitly described or claimed.

FIG. 1 is a block diagram of an example implementation of a fulfillment system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104. The system 100 may also include a storage device 110.

The benefit manager device 102 is a device operated by an entity that is at least partially responsible for creation and/or management of the pharmacy or drug benefit. This benefit may need to be tied or connected to the correct person or patient receiving the medication. Therefore, ensuring that the correct records are matched or not matched to the correct person or patient (as described herein) can be useful in proper operation of the fulfillment system 100. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long-term care benefit, a nursing home benefit, etc. The PBM may, in addition to its PBM operations, operate one or more pharmacies. The pharmacies may be retail pharmacies, mail order pharmacies, etc. The PBM may implement the record matching system as described herein.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in the storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the pharmacy system 100) following performance of at least some of the operations described above.

As part of the adjudication, a plan sponsor (or the PBM on behalf of the plan sponsor) reimburses the pharmacy for filling the prescription drug when the prescription drug was successfully adjudicated. The adjudication operations described above generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Virginia. Although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfilment device 112 to fulfill a prescription and dispense prescription drugs. In some implementations, the order processing device 114 may be an external order processing device separate from the pharmacy and in communication with other devices located within the pharmacy. The order processing device 114 may refer to information contained in a patient record to determine which medication and/or how much medication is to be provided.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as the order is fulfilled by the pharmacy fulfillment device 112. The prescription order may include one or more prescription drugs to be filled by the pharmacy. The order processing device 114 may make pharmacy routing decisions and/or order consolidation decisions for the prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor or more than one processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, and/or plan sponsor data 128. Further, the system 100 may include additional devices, which may communicate with each other directly or over the network 104.

The order data 118 may be related to a prescription order and may be obtained from a patient record. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM and may be obtained from one or more patient records. The information stored as member data 120 may include personal information, personal health information, protected health information, etc. Examples of the member data 120 include name, address, telephone number, e-mail address, prescription drug history, etc. The member data 120 may include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the use of the terms “member” and “user” may be used interchangeably.

The claims data 122 includes information regarding pharmacy claims adjudicated by the PBM under a drug benefit program provided by the PBM for one or more plan sponsors. The claims data 122 may be obtained from one or more patient records. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122. In some implementations, the claims data 122 includes claims that identify the members with whom the claims are associated. Additionally or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may be obtained from one or more patient records and can include information associated with a single medication or multiple medications. As used herein, the term common can be used to indicate that two or more things are the same. For example, a common name may be the same name, a common household may be the same household, and so on.

The prescription data 126 may include information regarding prescriptions that may be issued by prescribers on behalf of users, who may be members of the pharmacy benefit plan—for example, to be filled by a pharmacy. The prescription data 126 may be obtained from one or more patient records. Examples of the prescription data 126 include usernames, medication, or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.). In some implementations, the order data 118 may be linked to associated member data 120, claims data 122, drug data 124, and/or prescription data 126. The plan sponsor data 128 may be obtained from one or more patient records and can include information regarding the plan sponsors of the PBM. Examples of the plan sponsor data 128 include company name, company address, contact name, contact telephone number, contact e-mail address, etc.

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping. The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be in the same area or in different locations. For example, the devices 206-232 may be in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among several discrete devices and/or combined with other devices.

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may be comprised of order components. The order processing device 114 may use information contained in one or more patient records that are matched or not matched to the same patient to perform the operations described herein.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, etc. The order tracking subsystem 306 may store data locally (for example, in a memory) or as a portion of the order data 118 stored in the storage device 110.

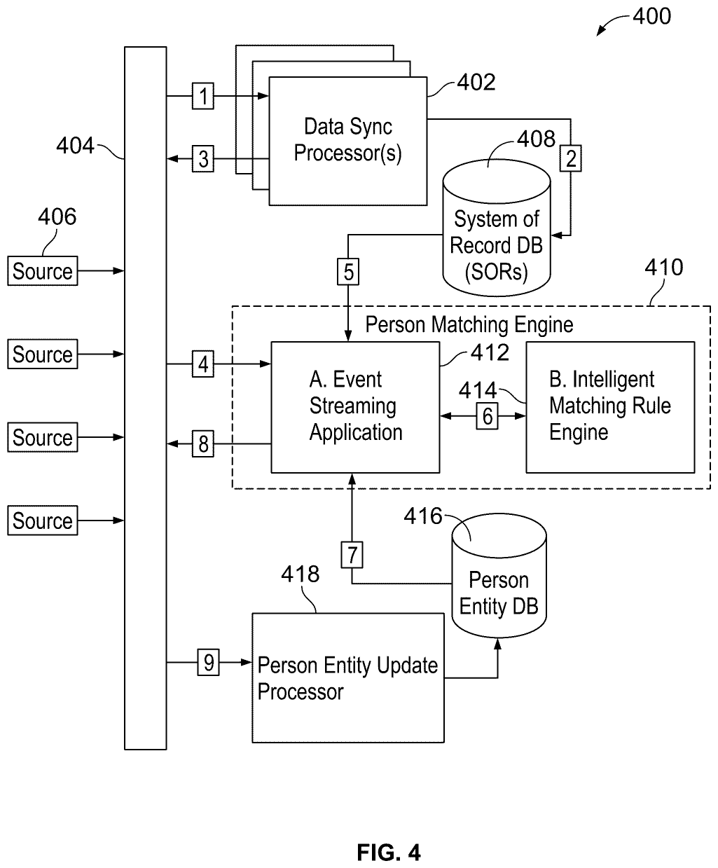

FIG. 4 illustrates one example of a patient record matching system 400. The record matching system 400 can unify data described herein to unify the data and associate different records with the same person or patient or determine that the records are not associated with the same person or patient to resolve patient identity. The record matching system 400 may use a person matching engine (PME) to efficiently perform person entity resolution using intelligent patient matching algorithms over distributed computing of the record matching system 400. Resolution of different entities (e.g., determining whether patient records match or do not match to the same person) can be an event driven Apache Spark Complex Event Processing (CEP) application in one example. This application can be designed to perform entity resolution in real-time or near real-time (e.g., delayed by unintentional or unavoidable processing delays). This application can handle large data volumes, as described above, and can be highly scalable and can be boosted by adding more worker nodes to the record matching system 400 to handle larger volumes of data and/or improve performance throughputs. The record matching system 400 may use an intelligent matching rule engine that can examine details of the records to determine whether two or more records include information representative of the same person (e.g., the records match each other) or different persons (e.g., the records do not match each other). Records that match each other may include different information (e.g., data from different healthcare providers), but information that is for or comes from examination of the same person. Records that do not match each other may include different information (e.g., data from different healthcare providers), but information that is not for or does not come from examination of the same person.

The matching rule engine can be implemented using a drools business rule management system (BRMS) or another type of system. The matching rule engine can be used to create (e.g., author) and manage the rules used for efficient and intelligent record matching. The matching rule engine can be used for independent rule authoring, testing, building, and deploying for matching rule consumption at runtime. For example, while the record matching system 400 is operating to examine and determine whether various records match to the same person, the matching rule engine can be used to write (e.g., create) and test new rules for use in determining whether records match each other, without having to take down or deactivate the ongoing examination of records using existing (e.g., previously written, tested, and approved) rules. This can allow for continuous or near continuous examination of records for matching while new rules are created and/or tested. The rules may rely on identifying relationships between information in different records, name pattern matching, entity locational patterns, and supervised learning.

The record matching system 400 includes one or more synchronization processors 402 that can examine a stream 404 of messages communicated from one or more sources of the records. The stream 404 can represent messages communicated to and/or from the sources of the records in the network 104 (shown in FIG. 1). Optionally, the stream 404 can represent the network 104. The synchronization processors 402 can represent one or more microprocessors, controllers, integrated circuits, field programmable gate arrays, or the like, that individually or collectively perform the operations described herein. For example, each synchronization processor 402 can perform all the operations attributed to the synchronization processors 402, or the operations attributed to the synchronization processors 402 may be divided up among the synchronization processors 402 so that two or more different processors 402 can perform different ones of the operations.

The synchronization processors 402 can examine or listen for messages (e.g., Kafka messages or another type of message) from different sources 406 of information for medical records. These sources 406 can represent healthcare facilities, pharmacies, insurance benefit providers, PBMs, or the like, that create and/or modify medical records of patients. The synchronization processors 402 may receive and/or examine messages sent from the sources 406 (e.g., to each other, to the processors 402, to electronic medical record (EMR) or system of records (SOR) databases 408 or storages having servers that store the records, or the like), where these messages may include new records, information updated in the records, changes to the records, or the like. The messages may be created whenever a record is created or updated in one example. The synchronization processors 402 can listen or look for these messages as the messages are communicated (e.g., and not only examine the messages in batches). With respect to the SOR database, the record matching system 400 may include one or more (or many) of the SOR databases 408.

The SOR databases 408 can represent tangible and non-transitory computer readable storage media, or computer memories, that store data. The SOR databases 408 can store current or the most up-to-date versions of patient records from the different sources 406. Because different sources 406 may provide different records for the same person, the SOR databases 408 may store many different records for the same person (and for different persons). In one example, the record matching system 400 may include a different SOR database 408 for each of the sources 406, with the SOR database 408 for each source 406 storing the most recent or most current record for that person from that source 406. Alternatively, a single SOR database 408 may store the records from two or more sources 406.

Responsive to detecting or identifying an update to a record or a new patient record from a source 406, the synchronization processor(s) 402 can send an update message to one or more of the sources 406 to indicate that the synchronization processor(s) 402 received the updated record or the new record. A person matching engine 410 of the matching system 400 can examine the messages sent to and/or from the synchronization processors 402 (e.g., to and/or from the SOR database(s) 408) to identify when a record is created or updated by one or more of the sources 406. The person matching engine 410 can represent one or more processors (e.g., similar to the synchronization processors 402) or another computing system. For example, the person matching engine 410 may be one or more software applications implemented by one or more processors. The person matching engine 410 can examine the messages communicated by the sources 406 via the stream 404 or network to determine when a record is updated or created, similar to the synchronization processor(s) 402. This can occur in parallel with the synchronization processors 402 monitoring the stream 404 for new messages.

The person matching engine 410 can include or operate an event streaming application 412 (e.g., a software application) that can monitor the stream 404 for new or updated records, obtain the updated or new record from the stream 404, and identify and obtain potentially matching records from the SOR database(s) 408 (as described herein). These potentially matching records may be records that may differ from the updated or new record from the stream 404, but that may potentially belong or be associated with the same person. These potentially matching records may be referred to as suspect or suspected records.

The person matching engine 410 also may include or may operate an intelligent matching rule engine 414, which can represent another software application. The updated or new record and the suspected records may be sent from the event streaming application 412 to the intelligent matching rule engine 414. The rule engine 414 can obtain a person entity profile from a person entity database 416 of the matching system 400. The person entity database 416 can store different profiles of persons having records from the sources 406. These profiles may contain demographic information of the persons, such as names, birth dates, addresses, genders, or the like. Several profiles may be generated for the same person. For example, each of the sources 406 may create a profile of a person, and the profile from each source 406 may contain or be associated with a source identifier associated with that source 406.

The rule engine 414 can examine information contained in the suspect records and the new/updated record and decide whether the new/updated record matches one or more of the suspect records, whether to merge or combine the new/updated record with one or more of the suspect records (to create a merged record as the records are associated with the same person, and optionally eliminate the new/updated record or the one or more suspect records), or whether to split the records (e.g., keep the new/updated record separate from the suspect record(s) due to the new/updated record and the suspect record(s) being associated with different persons).

The matching system 400 can include one or more implementation processors 418. The implementation processors 418 can represent one or more microprocessors, controllers, integrated circuits, field programmable gate arrays, or the like, that individually or collectively perform the operations described herein. The implementation processors 418 can implement actions based on recommendations or decisions output by the person matching engine 410. For example, the implementation processors 418 can listen to the outcomes of the person matching engine 410 via the stream 404. These outcomes can be decisions by the person matching engine 410 to merge records together or to split the records apart (or keep the records separate). The implementation processors 418 can then merge or split the records accordingly.

This process of operation of the record matching system 400 can operate in real time or near real time as records are updated or created and communicated via the stream 404. This can allow for near real time updating and merging of separate records that are identified as belonging to or being associated with the same person, as well as keeping records having information of different persons separate, to ensure that patients are receiving the correct medical care and medications (when decisions related to treatment or medications are based on the records).

Operation of the person matching engine 410 may involve at least two independent operations or steps that are completely decoupled from each other throughout the lifecycle of each operation or step. For example, the performance of one operation or step of the matching engine 410 may not depend on or be impacted by the performance of another operation or step of the matching engine 410, as described herein. These operations or steps may operate separately from each other in one embodiment.

One of these operations or steps includes a matching business rule authoring step. This can include designing, testing, implementing, lifecycle managing, archiving, and controlling rules or criteria used to determine whether records are to be merged with each other or split (kept separate) from each other. Designing the rules can involve creating or writing new rules. Implementing the rules can involve uploading the new or modified rules into the rule matching engine 414 identifying matched records, for merging records, for splitting records, or the like.

Testing the rules can involve using the new or modified rules to determine whether to merge or split records, but before the rules are used to actually merge or split the records. For example, the new or modified rules can be used by the matching rule engine 414 to determine whether records are to be merged or split (and the results of that determination can be output to a user, such as via a display device), but without the records actually being merged or split. This can allow the user to determine whether the rules are overmatching the records (e.g., determining that records match when the records are not associated with the same person) or undermatching the records (e.g., determining that records do not match when the records are associated with the same person) before the rules are used to actually change the records (e.g., by merging the records).

Implementing the rules can involve uploading the new or modified rules that have been tested into the rule matching engine 414 for merging or splitting records. Lifecycle managing can involve examining the rules that are being used for merging or splitting the records to determine whether the records need to be updated, discarded, or replaced with a new or different rule. Archiving the rules can involve storing records that were previously used for merging or splitting records (e.g., in one or more of the databases described herein). Optionally, archiving the rules can involve storing records that were tested, but not implemented in merging or splitting records. Controlling the rules can involve determining which rules are used for determining whether to split or merge records.

The authoring step can allow for users of the rule matching system 400 to design and write rules that compare different features or information contained in different records to determine whether the records are associated with the same person or with different persons. As one example, a rule can determine that records are associated with the same person if the records include demographic information (e.g., last names, social security numbers, mailing addresses, zip codes, phone numbers, identification numbers, insurance, or other benefit number, etc.) that match at least a designated percentage or amount. This rule or another rule can determine that records are not associated with the same person if the records include demographic information that does match at least the designated percentage or amount.

The rule matching system 400 can continue (a) operating to identify suspect records, (b) determining whether the suspect records match a new or updated record, and (c) either merging the records or keeping the records separate from each other based on that determination while the rules used in the determining operation are created and/or modified. Stated differently, the rules may be created and/or modified (and tested, as described herein) while the rule matching system 400 continues to merge records or keep the records separate (using existing rules that are not in the process of being modified or created). In one embodiment, an enterprise git and continuous integration (CI/CD) framework can be used for version control of the rules and deployment of the rules.

Another independent operation or step of the person matching engine 410 can involve an event streaming application execution step. This step can include designing the event streaming application 412, as well as testing, archiving, and source control of the event streaming application 412. For example, similar to the rule authoring step or operation, the streaming application 412 can involve the creation, testing, and use of rules to identify which records are suspect records. These rules can be created or modified and tested with the results being presented to a user, without interruption of the event streaming application 412 actually identifying suspect records for the matching rule engine 414 and without any downtime of the event streaming application 412.

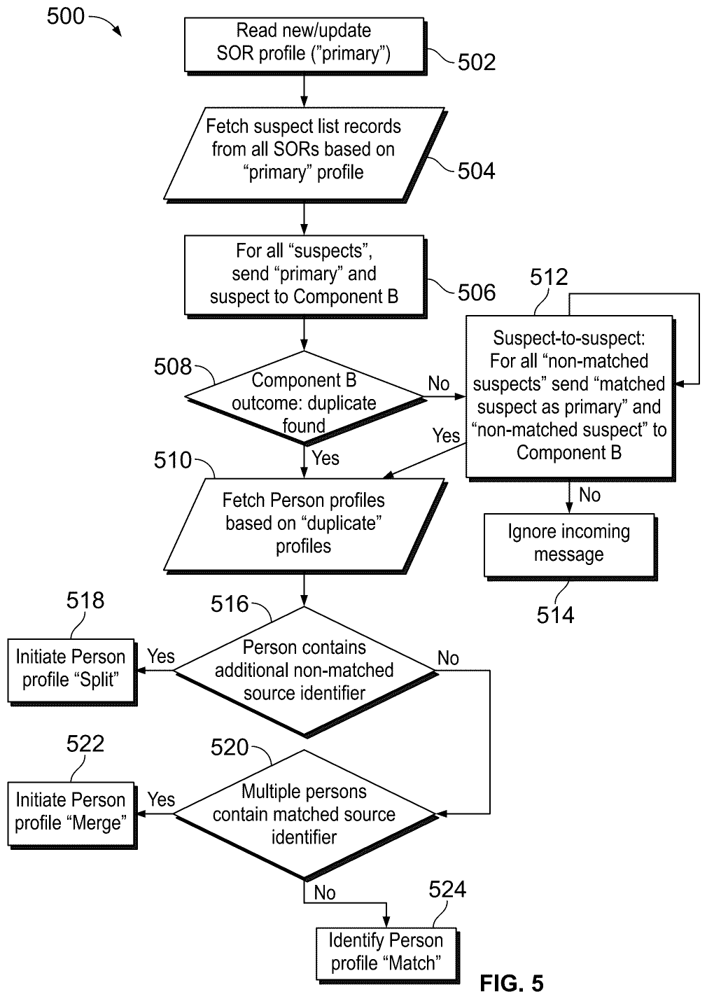

FIG. 5 illustrates a flowchart of one example of a method 500 for determining whether records match each other. The method 500 can represent operations performed by the record matching system 400 shown in FIG. 4. At 502, a new or updated SOR profile is read. This can involve the event streaming application 412 shown in FIG. 4 identifying a new or updated record in the stream 404 shown in FIG. 4.

At 504, one or more suspect records are obtained. The suspect record(s) can be fetched by the event streaming application 412 from the SOR database(s) 408. In one example, the event streaming application 412 can identify and fetch the suspect record(s) as the record(s) having (a) the same first name or last name and (b) birth date as the new or updated record, having (c) the same first name or last name and (d) phone number as the new or updated record, having (e) the same first name or last name and (f) the same postal address as the new or updated record, having the same social security number as the new or updated record, or having the same source identifier as the new or updated record. A source identifier may be a unique identity, code, numeric string, alphabetic string, alphanumeric string, or the like, that a source 406 may associate or include in records generated by the source 406. For example, each source 406 (or at least one or more of the sources 406) may include a unique identifier in each record, with the identifier uniquely identifying or associated with a single person. The source 406 can include this identifier in records from that source 406 to assist that source 406 in associating records of the same person with that person. But different sources 406 may use different identifiers for the same person as there may not be any universally unique identifier for each person used by all the sources 406.

At 506, the suspect and primary records are sent to the matching rule engine 414. For example, the new or updated record obtained by the event streaming application 412 and the suspect records identified by the event streaming application 412 are provided to the matching rule engine 414. At 508, a decision is made as to whether the new/updated record is a duplicate of one or more of the suspect records. For example, the matching rule engine 414 uses one or more rules that define criteria by which contents of the new/updated record and the suspect record(s) are compared. If the contents of the new/updated record and the suspect record(s) satisfy or meet the criteria, then the matching rule engine 414 may decide that the new/updated record and the suspect record(s) match each other. As a result, flow of the method 500 can proceed toward 510. But if the contents of the new/updated record and the suspect record(s) do not satisfy or meet the criteria, then the matching rule engine 414 may decide that the new/updated record and the suspect record(s) do not match each other. As a result, flow of the method 500 can proceed toward 512.

At 512, if two or more suspect records were identified and obtained (e.g., at 504), then the matching rule engine 414 may compare contents of the suspect records with each other to decide whether any two or more of the suspect records match each other. The matching rule engine 414 may use the same rule(s) to compare the suspect records with each other as were used to compare the new/updated record (e.g., the primary record) with the suspect record(s). Alternatively, the matching rule engine 414 may use one or more other, different rules to decide whether the suspect records match each other. If two or more suspect records are found to match each other, then flow of the method 500 can proceed toward 510. But if no suspect records are identified as matching each other, then flow of the method 500 can proceed toward 514. At 514, the message containing the new/updated record can be ignored. For example, the matching rule engine 414 can decide that the new/updated record and the suspect record(s) are not associated with the same person and that the suspected records (if there are two or more) are not associated with the same person. Flow of the method 500 can then terminate or return to a prior operation, such as 502.

At 510, the records that were found to match each other are compared with the entity profiles. For example, the matching engine 414 can compare demographic information in the records found to match each other at 508 with the corresponding profiles associated with those records in the entity database 416. At 516, if the source identifiers in the records that were found to match each other at 508 are also present in a single or multiple person entity profiles in the entity database 416 along with one or more other, different source identifiers that do not match the source identifiers in the matched records, then a decision may be made to split the profiles in the profile entity database 416 at 518. For example, the matching rule engine 414 may separate the profiles in the database 416.

But, if the source identifiers in the matching records are not present in a single or multiple person entity profiles in the entity database 416 along with other, different source identifiers that do not match the source identifiers in the matched records, then flow of the method 500 can proceed toward 520. At 520, a decision is made as to whether the source identifiers in the matched records are present in multiple, different entity profiles in the entity database 416. If the source identifiers in the matched records are in multiple, different entity profiles in the entity database 416, then these profiles may be merged into a single profile in the entity database 416 at 522. For example, the information in these two or more profiles may be combined into a single profile in the entity database, with the additional profiles removed from the database 416 after being merged. In an example embodiment, the profiles are not removed but the merged profiles are flagged as inactive, merged, split, or not-for-production in the database. These not use records can be used for auditing, analytics or other historical purposes. Flow of the method 500 may return to a prior operation, such as 502.

But, if all the source identifiers in the matched records are exactly present in a single entity profile in the profile database 414, then the matching rule engine 414 may decide at 520 that all these records perfectly match each other and are associated with the same person at 524. Flow of the method 500 can return to a prior operation, such as 502.

In any of the “split,” “merge,” and “match” decisions are made (e.g., the method 500 proceeds to 518, 522, or 524), then the new/updated record is found to match at least one of the suspect records, and can be used to conduct one or more medical decisions or operations, such as prescribing a medication, filling a prescription, or the like, for the person associated with the new/updated record and the suspect record(s).

Finally, if (a) the source identifiers in all matched records are not exactly present in a single entity profile (i.e., a match is not identified at 520, 524), (b) the source identifiers in the matched records are not present in multiple entity profiles (i.e., a merge is not identified at 520, 522), and (c) all source identifiers in the matched records are not present in a single or multiple entity profiles along with other source identifiers that are not in the matched records (i.e., a split is not identified at 516, 518), then the new/updated record received at 502 is found to not match any of the suspect records identified at 504. As a result, the new/updated record may not be identified as being associated with the same person as the suspect records, and the suspect records may not be used to conduct one or more medical decisions or operations, such as prescribing a medication, filling a prescription, or the like, for the person associated with the new/updated record.

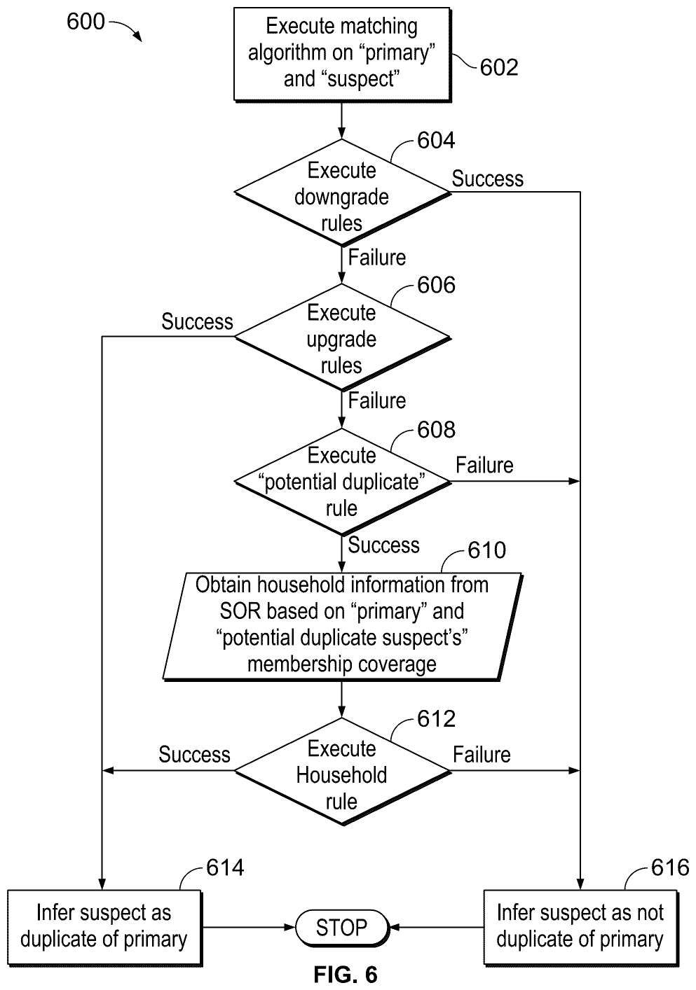

FIG. 6 illustrates one example of a flowchart for a method 600 of examining a new/updated record (e.g., a primary record) and one or more suspect records to determine whether these records match the same person or different persons. The method 600 can represent operations performed by the matching rule engine 414 to determine whether a new/updated record matches a suspect record. At 602, a matching algorithm is initiated on the records. This algorithm can be initiated by applying a series of rules or criteria to the information contained in the records to determine whether the information satisfies the rules or criteria are met or satisfied. If the rules or criteria are met or satisfied, the records may be determined to match each other. If the rules or criteria are not met or not satisfied, the records may be determined to not match each other. The information contained in the records that is compared using the rules can include names, birth dates, social security numbers or other external (non-source) identifiers, genders, postal addresses, phone numbers, benefit membership information, etc.

At 604, one or more downgrade rules are applied to the records. The downgrade rules may be rules that exclude records from matching each other. For example, the downgrade rules may compare the names in the records with the same benefit coverage and determine whether these names match. The benefit coverage may indicate an insurance provider listed in the records. If two records have the same benefit coverage, but different names (e.g., names that do not have at least a threshold number of consecutive characters that are the same, are not truncations of each other, are not designated nicknames of each other, etc.), then the matching rule engine 414 may determine that the records do not match each other. Flow of the method 500 may then proceed toward 616. As another example, if two records have similar names, addresses, social security numbers, (e.g., names, addresses, or social security numbers that have at least the threshold number of consecutive characters that are the same, are truncations of each other, or are designated nicknames of each other, etc.) and/or genders, but the birth dates in the records are more than a designated duration apart (e.g., the birth date in one record is at least ten years older or younger than the birth date in the other record), then the matching rule engine 414 may determine that the records do not match each other. Flow of the method 600 may then proceed toward 616. Otherwise, flow of the method 600 may proceed toward 606.

At 606, one or more upgrade rules are applied to the records. The upgrade rules may be rules that include records matching each other. For example, the upgrade rules may look for matching attribute combinations of information in the records being examined. One such rule may be used to determine whether the first name, last name, birth date, and postal address in the primary record matches the same information in the suspect record. Another rule may be used to determine whether the first name, birth date, and social security number in the primary record matches the same information in the suspect record. A rule may be used to determine whether the first name, social security number, gender, and phone number in the primary record matches the same information in the suspect record. Another rule may be used to determine whether the last name, birth date, social security number, gender, and phone number in the primary record matches the same information in the suspect record. Another rule may be used to determine whether the first name, last name, postal address, gender, birth date year, and birth date month in the primary record matches the same information in the suspect record. Another rule may be used to determine whether the first name, birth date, gender, and source identifier in the primary record matches the same information in the suspect record. Another rule may be used to determine whether the last name, birth date, social security number, gender, postal address, and source identifier in different records in the SOR database 408 match each other. Another rule may apply pattern matching by looking into the records for dummy or default social security numbers (e.g., 999-99-9999), many different profiles in the profile entity database 416 being associated with the same address (e.g., a dummy or default address, such as 123 Main Street), etc. This rule may be applied by excluding records having such dummy or default information from being matched to other records.

For example, if the primary and suspect records have the same first name, the same last name, the same birth date, and the same postal address, then the records may be identified as matching each other and flow of the method 600 can proceed toward 614. Otherwise, flow of the method 600 can proceed toward 608. If the first name, birth date, and social security number in the primary record matches the same information in the suspect record, then the records may be identified as matching each other and flow of the method 600 can proceed toward 614. Otherwise, flow of the method 600 can proceed toward 608. If the first name, social security number, gender, and phone number in the primary record matches the same information in the suspect record, then the records may be identified as matching each other and flow of the method 600 can proceed toward 614. Otherwise, flow of the method 600 can proceed toward 608. If the last name, birth date, social security number, gender, and phone number in the primary record matches the same information in the suspect record, then the records may be identified as matching each other and flow of the method 600 can proceed toward 614. Otherwise, flow of the method 600 can proceed toward 608. If the first name, last name, postal address, gender, birth date year, and birth date month in the primary record matches the same information in the suspect record, then the records may be identified as matching each other and flow of the method 600 can proceed toward 614. Otherwise, flow of the method 600 can proceed toward 608. If the first name, birth date, gender, and source identifier in the primary record matches the same information in the suspect record, then the records may be identified as matching each other and flow of the method 600 can proceed toward 614. Otherwise, flow of the method 600 can proceed toward 608. If the last name, birth date, social security number, gender, postal address, and source identifier in different records in the SOR database 408 match each other, then the records may be identified as matching each other and flow of the method 600 can proceed toward 614. Otherwise, flow of the method 600 can proceed toward 608.

At 608, 610, and 612, household information is obtained from the SOR database 408. The household information can be obtained by the matching rule engine 414 for those primary and suspect records found to match each other at 604, 606, and/or 608. The household information. One or more potential duplicate rules are applied to the primary and suspect records to determine whether these records are duplicates (e.g., exact copies) of each other. That is, the records are exactly the same and not different records containing information for the same person. The matching rule engine 414 can determine whether (a) the first name or a combination of the first several (e.g., five) characters of the first name and the gender in the records match each other, (b) the last name in the records matches each other, and (c) the birth date in the records matches each other, then dependent profiles for the records are obtained from the SOR database 408. The dependent profiles may be profiles under the same benefit coverage. If any of these dependent profiles are found to match each other, then the primary and suspect records are identified as matching each other at 612. Flow of the method 600 can proceed toward 614. Otherwise, flow of the method 600 can proceed toward 616.

At 616, the suspect record is identified as a match or duplicate of the primary record. For example, the records may be identified by the matching rule engine 414 as including information of or belonging to the same person. At 618, the suspect record is not identified as a match or duplicate of the primary record. For example, the records may be identified by the matching rule engine 414 as not including information of or belonging to the same person. Flow of the method 600 may then terminate or return to a prior operation, such as 602.

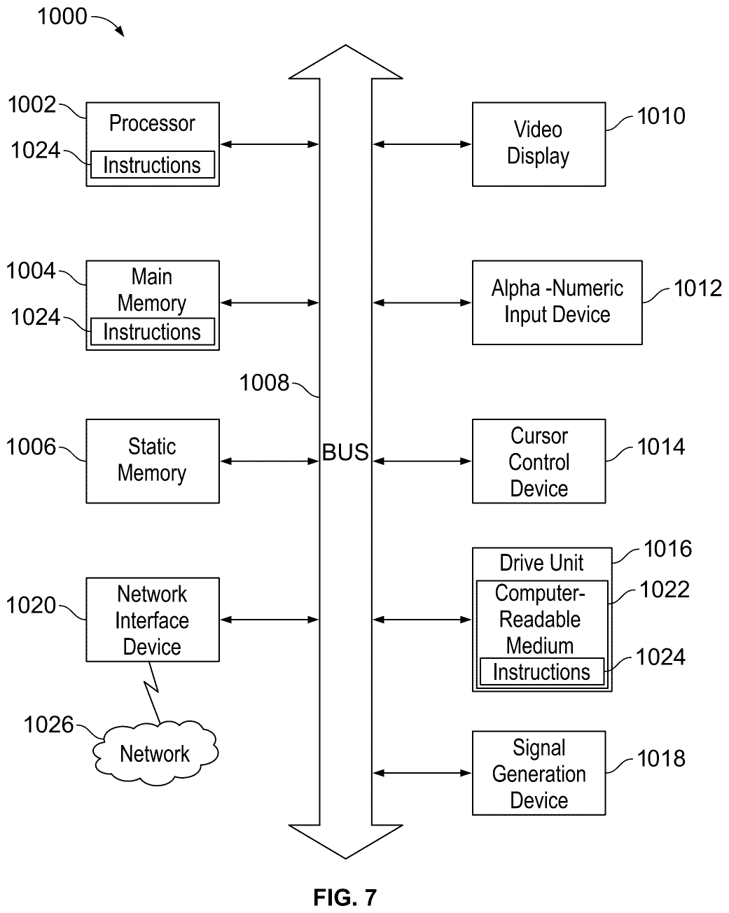

FIG. 7 shows a block diagram of a computer system 1000 within which a set of instructions may be executed causing the machine to perform any one or more than one methods, processes, operations, or methodologies discussed herein. For example, the system 1000 may represent the system 400 shown in FIG. 4, or at least the matching engine 410 shown in FIG. 4. The devices 1006-1030, for example, may include the functionality of the one or more than one computer systems 1000. These devices and systems are dedicated to performing any one or more than one methods, processes, operations, or methodologies discussed herein.

In an example embodiment, the machine operates as a standalone device or may be connected (e.g., networked, etc.) to other machines. In a networked deployment, the machine may operate in the capacity of a server or a client machine in server-client network environment, or as a peer machine in a peer-to-peer (or distributed) network environment.

The example computer system 1000 includes a processor or more than one processor 1002 (e.g., a central processing unit (CPU), a graphics processing unit (GPU) or both, etc.), a main memory 1004 and a static memory 1006, which communicate with each other via a bus 1008. The computer system 1000 further includes a video display unit 1010 (e.g., a liquid crystal display (LCD) or a cathode ray tube (CRT), etc.). The computer system 1000 also includes an alphanumeric input device 1012 (e.g., a keyboard, etc.), a cursor control device 1014 (e.g., a mouse, etc.), a drive unit 1016, a signal generation device 1018 (e.g., a speaker, etc.) and a network interface device 1020.

The drive unit 1016 includes a computer readable medium 1022 on which is stored one or more than one sets of instructions 1024 (e.g., software, etc.) embodying any one or more than one methodologies or functions described herein. The instructions 1024 may also reside, completely or at least partially, within the main memory 1004 and/or within the processor 1002 during execution thereof by the computer system 1000, the main memory 1004 and the processor 1002 also constituting non-transitory computer readable media. When loaded with the instructions 1024, the processor 1002 is a machine dedicated to only the present processes and methodologies. The instructions 1024 may further be transmitted or received over a network 1026 via the network interface device 1020. The network 1026 can represent the network 104 shown in FIG. 1.

While the computer-readable medium 1022 is shown in an example embodiment to be a single medium, the term “computer-readable medium” should be taken to include a single medium or multiple media (e.g., a centralized or distributed database, and/or associated caches and servers, etc.) that store the one or more than one sets of instructions. The term “computer-readable medium” shall also be taken to include any medium that is capable of storing or encoding a set of instructions for execution by the machine and that cause the machine to perform any one or more than one methodologies of the present disclosure. The term “computer-readable medium” shall accordingly be taken to include, but not be limited to, solid-state memories, and optical media, and magnetic media. In some embodiments, the computer-readable medium is a non-transitory computer-readable medium. In other examples, a computer-readable medium is any medium that satisfies statutory requirements and stores instructions for use by a machine.

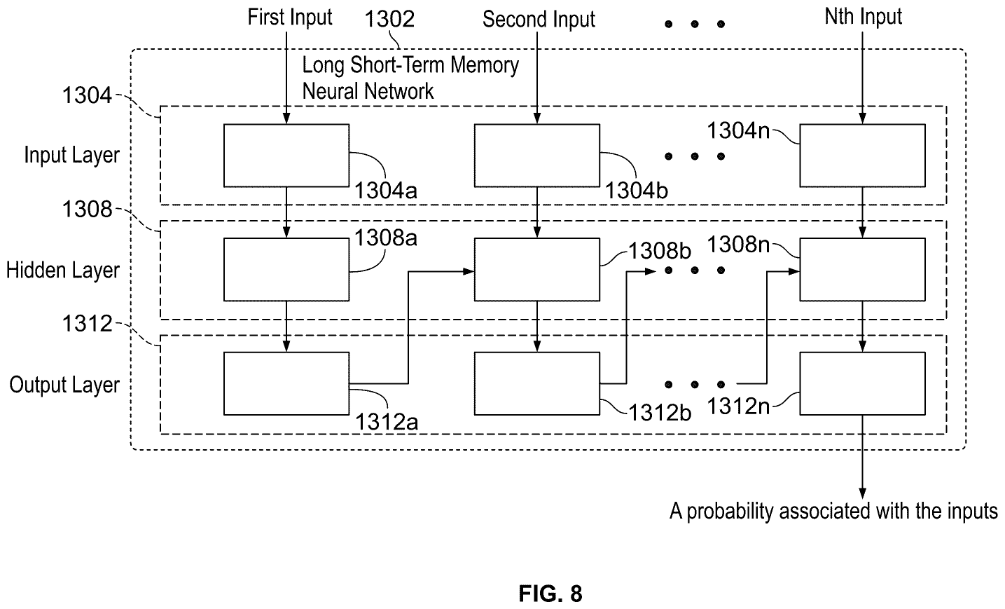

One or more of the record matching systems 400 described herein may be implemented in an AI or machine-learning system. FIG. 8 illustrates a functional block diagram of an example neural network 1302 that can be used by the record manager device for matching records with the same person as described herein. In an example, the neural network 1302 can represent a long short-term memory (LSTM) neural network. In an example, the neural network 1302 can represent one or more recurrent neural networks (RNN). The neural network 1302 may be used to implement the machine learning as described herein, and various implementations may use other types of machine learning networks. The neural network 1302 includes an input layer 1304, one or more intermediate or hidden layers 1308, and an output layer 1312. Each layer 1304, 1308, 1312 includes artificial individual units, or neurons. Each neuron can receive information (e.g., as input into the neural network 1302 or as received as output from another neuron in another layer or the same layer), process this information to generate output, and provide the output to another neuron or as output of the neural network 1302. The input layer 1304 may include several input neurons 1304a, 1304b . . . 1304n, the hidden layer 1308 may include several intermediate neurons 1308a, 1308b . . . 1308n, and the output layer 1312 may include several output neurons outputs 1312a, 1312b . . . 1312n. The inputs may include, for example, patient records, parts of patient records, or the like.

Each neuron can receive an input from another neuron and output a value to the corresponding output to another neuron (e.g., in the output layer 1312 or another layer). For example, the intermediate neuron 1308a can receive an input from the input neuron 1304a and output a value to the output neuron 1312a. Each neuron may receive an output of a previous neuron as an input. For example, the intermediate neuron 1308b may receive input from the input neuron 1304b and the output neuron 1312a. The outputs of the neurons may be fed forward to another neuron in the same or different intermediate layer 1308.

The processing performed by the neurons may vary based on the neuron but can include the application of the various rules or criteria described herein to partially or entirely decide whether two or more records match the same person or patient. The output of the application of the rule or criteria can be passed to another neuron as input to that neuron. One or more neurons in the intermediate and/or output layers 1308, 1312 can determine that the records match or do not match. The last output neuron 1312n in the output layer 1312 may output a matching or no-match decision. For example, the output from the neural network 1302 can be an indication that two (or more) different first names in the records match the same patient (and are nicknames), or that two (or more) different first names in the records do not match the same patient. Alternatively, the output can be a probability indicating that the records do (or do not) match to the same patient (and are not nicknames). Although the input layer 1304, the intermediate layer(s) 1308, and the output layer 1312 are depicted as each including three artificial neurons, one or more of these layers may contain more or fewer artificial neurons. The neurons can include or apply one or more adjustable parameters, weights, rules, criteria, or the like, as described herein, to perform the processing by that neuron.

In various implementations, the layers of the neural network 1302 may include the same number of artificial neurons as each of the other layers of the neural network 1302. For example, historical patient data may be processed to provide information to the input neurons 1304a-1304n. The output of the neural network 1302 may represent a match or no match of the records to the same patient. More specifically, the inputs can include known facts stored in the patient records. The known facts can be provided to the neurons 1308a-1308n for analysis and connections between the known facts. The neurons 1308a-1308n, upon finding connections, provides the potential connections as outputs to the output layer 1312, which can determine a record match, no record match, or a probability of a record match.

In some embodiments, the neural network 1302 may be a convolutional neural network. The convolutional neural network can include an input layer, one or more hidden or intermediate layers, and an output layer. In a convolutional neural network, however, the output layer may include one fewer output neuron than the number of neurons in the intermediate layer(s), and each neuron may be connected to each output neuron. Additionally, each input neuron in the input layer may be connected to each neuron in the hidden or intermediate layer(s).

Such a neural network-based record matching system can be trained by operators, automatically self-trained the record matching system itself, or can be trained both by operators and by the record matching system itself to improve how nicknames in the records are or are not matched with different first names in the patient records. This can allow for the record matching system to improve the accuracy with which records are (or are not) matched with each other over time to reduce instances and likelihoods of overmatching or undermatching records.

For example, the record matching systems described and shown herein may determine which different first names in the patient records belong to the same patient using one or more supervised machine-learning processes. These supervised machine-learning processes may include classification algorithms, defined as processes where the artificial neurons of the computer system derive, from training data, one or more sets of the matching rules described herein (e.g., one or more machine-learning models) for analyzing input records to determine whether two (or more) different names in the records match to the same person (or do not match to the same person).

The AI or machine-learning processes may be used to generate or update the machine-learning models used by the artificial neurons to compare information from the patient records. A machine-learning model can be or include a mathematical representation of a relationship between input records and outputs (e.g., indications of a match between records to the same person, indications of a match between different first names as nicknames of each other, indications of no match between records or different first names to the same person, or indications of no match between different first names as nicknames of each other), as generated using the machine-learning processes described herein. An input is provided to one or more of the input neurons of the neural network 1302 after the model is created. The output neurons within the network 1302 generate an output based on the relationships that are derived or learned by the neurons in the intermediate or hidden layer(s) 1308. Connections between the nodes within each layer and/or between the layers 1304, 1308, 1312 and/or the neurons may be created via the process of training the record matching network or record matching system. This training can adjust connections, values of parameters, weights, etc., between the neurons in the layers 1304, 1308, 1312 of the neural network 1302 to reduce instances of overmatching or undermatching records over time (and relative to another system that does not change the connections, parameter values, weights, etc.). Additionally or alternatively, the record matching system can train itself by adding or creating rules or criteria for analyzing records for matches. This repeated training process can be referred to as deep learning.

This training can involve, for example, providing data as input to the neurons in the input layer 1304 of the record matching system (e.g., patient records), and having the neurons in the input layer 1304 and/or intermediate layer(s) 1308 of the record matching system apply the matching rules, criteria, and/or processes described herein. This application of the rules, criteria, and/or processes is performed by the neurons in the intermediate layer(s) 1308 to determine (e.g., as output from the output neurons in the output layer 1312) whether two or more of the records (in the provided data) match to the same patient (or do not match to the same patient). This output (e.g., identifications of records matching or not matching to the same person) can then be examined to determine whether the output is correct or not (e.g., determining whether the records actually match to the same person, or determining whether the analysis by the system was incorrect). This analysis and decision can be performed by one or more of the neurons in the layers 1304, 1308, 1312 of the neural network 1302, or can be manually performed (e.g., as a check on operation of the neural network 1302).

For erroneous matches or non-matches that are output by the record matching system, the matching rules, criteria, and/or processes used by one or more of the neurons in the record matching system can then be modified, augmented, or reduced. The rules or criteria can be modified by changing values of parameters, weights, thresholds, volumes, etc. (as described herein), may be augmented by adding one or more rules or criteria that were not previously used to examine the records, and/or may be reduced by eliminating one or more rules or criteria that were previously used to examine the records. The same and/or different data (e.g., patient records) can subsequently be input into the input neurons in the input layer 1304, and the process repeated. For example, the output of the neurons in the output layer 1312 after the modification can be examined to determine whether the identified matches or non-matches are correct, with the matching rules, criteria, and/or processes used by the neurons in the intermediate layer(s) 1308 potentially modified again. This process can be repeated several times to improve the accuracy by which the record matching system matches (or does not match) records to each other. For example, the output(s) provided by the output neurons in the output layer 1312 can be examined to determine whether any overmatching or undermatching occurred. If any overmatching or undermatching did occur, then the processes performed by one or more of the neurons in the intermediate layer(s) 1308 can be modified as described herein. The process can then be repeated to gradually reduce instances of overmatching and/or undermatching.

In contrast to some known artificial intelligence or machine learning systems that use supervised learning to train the systems or networks, one or more embodiments of the neural network 1312 of the record matching systems described herein may use unsupervised learning to train the systems. For example, actual patient records may be used as the production data that is input into the input neurons of the neural network 1302 for analysis and identification of matches by the neurons in the intermediate layer(s) 1308. These patient records may not be known to match (or not match) each other prior to inputting the records into the record matching system. Instead, the output from the neurons in the output layer 1312 record matching system can be examined to determine whether the system-matched records (or records that are not identified as matches to the same person) are correct matches of records, incorrect matches of records, or missed matching of records (e.g., where two or more records are associated with the same person, but the system does not identify the records as matching). Optionally, field experience may be used for direct machine learning. Healthcare providers may learn about individual undermatches and overmatches with records and correct these records. In one embodiment, the matching system can use these manual corrections to learn and infer refinements to parameters and rules.

The analysis of the output can be performed by receiving output from the neuron(s) in the output layer 1312 indicating that two (or more) records match the same patient; examining the matched records to confirm or refute that the records do, in fact, belong to the same patient; and generating feedback based on this examination. For example, the matched records may be used to provide a medical decision, such as filling a prescription, making a medical diagnosis, etc. The same or another system (e.g., a pharmacy benefit manager device, a physician's office, a hospital records system, a clinical information system, etc.) can determine (based on additional analysis of the matched records) whether the matched records do, in fact, belong to the same person. Alternatively, these other systems can include one or more persons that manually inspect the matches records to determine whether the records do contain medical data of the same person or different persons.

If these other automated and/or manual systems determine that the records matched by the record matching system do not match the same person (e.g., the records belong to different persons), then feedback data can be generated and provided back to the record matching systems. For example, the feedback data can be provided as input to one or more of the neurons in the input layer 1304 of the network 1302. This feedback data can identify mis-matches in the records. For example, a mismatch can be identified a binary match or no-match indication, can be identified by a subset of the data within the records that was found to indicate the records do not belong to the same person, or the like.

The record matching system can then change (or have changed) one or more parts of the matching rules or criteria used by the neurons in the intermediate layer(s) 1308 to examine the records, as described above. Weights or other parameters of the matching rules or criteria used by these neurons may be modified to train the record matching system to not overmatch or undermatch the records going forward. The record matching system can change the number of instances where records show different names in the same household but that share a unique demographic marker in at least a certain threshold proportion of households and/or volume of households in which the names co-exist. For example, based on the feedback data indicating overmatching or undermatching of records, the record matching system can change the threshold proportion of households and/or volume of households to reduce overmatching and/or undermatching. If the proportion of instances where the records show the different names in the same household with the same demographic marker is no greater than the threshold that is changed based on the feedback, then the record matching system can determine that there is not enough confidence to determine that the different names are nicknames. But if the proportion and/or volume of instances where the records show the different names with the same demographic marker is greater than the threshold that has been changed due to the feedback, then the record matching system can determine that there is enough confidence to determine that the different names are nicknames. Over time, this learning process can improve identification of common nicknames to improve the accuracy by which the record matching system identifies when records match to the same person.

The record matching system may train itself or be trained based on the feedback data by adding and/or removing rules, criteria, or parameters. For example, during one analysis of records, the record matching system may use a first set of the rules, criteria, and/or parameters described herein. Based on feedback indicating that the record matching using this first set of rules, criteria, and/or parameters including overmatching or undermatching, the recording matching system may train itself (or be trained) by using a second set of rules, criteria, and/or parameters for future analysis of the same and/or different records. This second set may include one or more additional rules, criteria, and/or parameters that were not in the first set during at least one previous iteration of record analysis. Additionally or alternatively, second set may include one or more fewer rules, criteria, and/or parameters that were in the first set during at least one previous iteration of record analysis.

The record matching systems and methods described herein provide a technical improvement over conventional record matching techniques by significantly reducing the complexity of manually matching records with each other in a timely manner (e.g., in the time period where medical decisions need to be made based on the records, such as within several minutes but less than an hour). The complexity of matching the records can be reduced significantly because the only activity required from human users for record-matching purposes is to use the information from the records that are matched for making one or more medical decisions at a suitable timing or within a suitable time period for the patient. Compared to prior art and conventional record matching techniques, the subject matter described herein matches records in less time, with less user interaction, and with fewer errors.

One or more embodiments of the subject matter described herein involve a computer's utilization of a machine learning or statistical model to analyze many medical records received from multiple, different sources to determine which records match each other or include information for the same person, regardless of errors or mismatches between identifying demographic information that otherwise would result in the records being incorrectly matched to the same person or incorrectly matched to different persons, thereby posing significant and deadly risks to patients. While some examination of medical records can be performed mentally or with pen and paper, the subject matter described herein may transform (through training or machine learning) a neural network and data contained in the medical records to determine whether records match the same or different persons.

In one example, a method includes monitoring a stream of messages from plural different sources of medical records to identify a new or updated record of the medical records, and identifying one or more suspect records from an SOR database. The one or more suspect records are identified as being potential matches to the new or updated record that is identified. The method also includes determining that the one or more suspect records match the new or updated record, obtaining a person entity profile related to the new or updated record from a person entity database, and merging the new or updated record with the one or more suspect records or splitting the new or updated record from the one or more suspect records based on the person entity profile that is obtained.

The new or updated record can be identified in real time. The one or more suspect records can be determined to match the new or updated record by comparing information contained within the one or more suspect records and the new or updated record using matching rules.

The method also can include authoring one or more new rules or one or more changes to the matching rules off-line while the matching rules without the one or more new rules or the one or more changes continue to be used to determine whether the one or more suspect records match the new or updated record. The method may also include testing the one or more new rules or the one or more changes to the matching rules off-line using the medical records without merging the new or updated record with the one or more suspect records and without splitting the new or updated record from the one or more suspect records.

The one or more suspect records can include plural suspect records, and the method also can include determining that two or more of the suspect records match each other, and merging the two or more of the suspect records with each other. The medical records can include source identifiers uniquely identifying which of the sources generated the medical records and the person entity database stores the source identifiers associated with each of several different persons. Merging the new or updated record with the one or more suspect records can occur responsive to the source identifiers in the new or updated record and the one or more suspect records being associated with two or more of the different persons in the person entity database.

The medical records can include source identifiers uniquely identifying which of the sources generated the medical records and the person entity database stores the source identifiers associated with each of several different persons. Splitting the new or updated record from the one or more suspect records can occur responsive to the source identifiers in the new or updated record and the one or more suspect records being associated with a single person of the different persons in the person entity database along with one or more other source identifiers.

In one example, a record matching system includes one or more data sync processors configured to monitor a stream of messages from plural different sources of medical records to identify a new or updated record of the medical records, and a person matching engine configured to identify one or more suspect records from an SOR database. The one or more suspect records are identified as being potential matches to the new or updated record that is identified. The person matching engine also is configured to determine that the one or more suspect records match the new or updated record, obtain a person entity profile related to the new or updated record from a person entity database, and merge the new or updated record with the one or more suspect records or splitting the new or updated record from the one or more suspect records based on the person entity profile that is obtained.

The one or more data sync processors can identify the new or updated record in real time. The person matching engine can determine that the one or more suspect records match the new or updated record by comparing information contained within the one or more suspect records and the new or updated record using matching rules. The person matching engine can be used to author one or more new rules or one or more changes to the matching rules off-line while the matching rules without the one or more new rules or the one or more changes continue to be used to determine whether the one or more suspect records match the new or updated record.

The person matching engine can test the one or more new rules or the one or more changes to the matching rules off-line using the medical records without merging the new or updated record with the one or more suspect records and without splitting the new or updated record from the one or more suspect records. The one or more suspect records can include plural suspect records, and the person matching engine can determine that two or more of the suspect records match each other. The system also can include one or more update processors that merge the two or more of the suspect records with each other.

The medical records can include source identifiers uniquely identifying which of the sources generated the medical records and the person entity database stores the source identifiers associated with each of several different persons. The person matching engine can merge the new or updated record with the one or more suspect records responsive to the source identifiers in the new or updated record and the one or more suspect records being associated with two or more of the different persons in the person entity database.

The medical records can include source identifiers uniquely identifying which of the sources generated the medical records and the person entity database stores the source identifiers associated with each of several different persons. The person matching engine can split the new or updated record from the one or more suspect records responsive to the source identifiers in the new or updated record and the one or more suspect records being associated with a single person of the different persons in the person entity database along with one or more other source identifiers.

In another example, a method includes monitoring a stream of messages from plural different sources of medical records to identify a new or updated record of the medical records, and identifying one or more suspect records from an SOR database. The one or more suspect records are identified as being potential matches to the new or updated record that is identified. The method also includes determining that the one or more suspect records match the new or updated record, and obtaining a person entity profile related to the new or updated record from a person entity database. The person entity profile includes source identifiers uniquely identifying which of the sources generated the medical records associated with each of several different persons. The method also includes (a) merging the new or updated record with the one or more suspect records and/or (b) splitting the new or updated record from the one or more suspect records. Merging the new or updated record with the one or more suspect records occurs responsive to the source identifiers in the new or updated record and the one or more suspect records being associated with two or more of the different persons in the person entity database. Splitting the new or updated record from the one or more suspect records occurs responsive to the source identifiers in the new or updated record and the one or more suspect records being associated with a single person of the different persons in the person entity database along with one or more other source identifiers.

The new or updated record can be identified in real time. The one or more suspect records can be determined to match the new or updated record by comparing information contained within the one or more suspect records and the new or updated record using matching rules. The method also can include authoring one or more new rules or one or more changes to the matching rules off-line while the matching rules without the one or more new rules or the one or more changes continue to be used to determine whether the one or more suspect records match the new or updated record. The method also can include testing the one or more new rules or the one or more changes to the matching rules off-line using the medical records without merging the new or updated record with the one or more suspect records and without splitting the new or updated record from the one or more suspect records.

The present description, in various embodiments, provides a novel data processing algorithm that when implemented, e.g., on a machine dedicated to running the instructions for the methodologies described herein, improves data processing.

In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, present disclosure may lie in less than all features of a single disclosed embodiment. Thus, the following claims are hereby incorporated into the Detailed Description, with each claim standing on its own as a separate embodiment.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, demonstrates the flow of information (such as data or instructions) that is of interest to the illustration. For example, when element A and element B exchange a variety of information, but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

None of the elements recited in the claims are intended to be a means-plus-function element within the meaning of 35 U.S.C. § 112(f) unless an element is expressly recited using the phrase “means for,” or in the case of a method claim using the phrases “operation for” or “step for.”

The methods described herein do not have to be executed in the order described, or in any particular order. Moreover, various activities described with respect to the methods identified herein can be executed in serial or parallel fashion. Although “End” blocks may be shown in the flowcharts, the methods may be performed continuously.

In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, present disclosure may lie in less than all features of a single disclosed embodiment. Thus, the following claims are hereby incorporated into the Detailed Description, with each claim standing on its own as a separate embodiment.

What is claimed is:

1. A method comprising:
monitoring a stream of messages from plural different sources of medical records to identify a new or updated record of the medical records;
identifying one or more suspect records from a system of record (SOR) database, the one or more suspect records identified as being potential matches to the new or updated record that is identified;
determining that the one or more suspect records match the new or updated record;
obtaining a person entity profile related to the new or updated record from a person entity database; and
merging the new or updated record with the one or more suspect records or splitting the new or updated record from the one or more suspect records based on the person entity profile that is obtained.

2. The method of claim 1, wherein the new or updated record is identified in real time.

3. The method of claim 1, wherein the one or more suspect records are determined to match the new or updated record by comparing information contained within the one or more suspect records and the new or updated record using matching rules.

4. The method of claim 3, further comprising:
authoring one or more new rules or one or more changes to the matching rules off-line while the matching rules without the one or more new rules or the one or more changes continue to be used to determine whether the one or more suspect records match the new or updated record.

5. The method of claim 4, further comprising:
testing the one or more new rules or the one or more changes to the matching rules off-line using the medical records without merging the new or updated record with the one or more suspect records and without splitting the new or updated record from the one or more suspect records.

6. The method of claim 1, wherein the one or more suspect records include plural suspect records, and further comprising:
determining that two or more of the suspect records match each other; and
merging the two or more of the suspect records with each other.

7. The method of claim 1, wherein the medical records include source identifiers uniquely identifying which of the sources generated the medical records and the person entity database stores the source identifiers associated with each of several different persons,
wherein merging the new or updated record with the one or more suspect records occurs responsive to the source identifiers in the new or updated record and the one or more suspect records being associated with two or more of the different persons in the person entity database.

8. The method of claim 1, wherein the medical records include source identifiers uniquely identifying which of the sources generated the medical records and the person entity database stores the source identifiers associated with each of several different persons,
wherein splitting the new or updated record from the one or more suspect records occurs responsive to the source identifiers in the new or updated record and the one or more suspect records being associated with a single person of the different persons in the person entity database along with one or more other source identifiers.

9. A record matching system comprising:
one or more data sync processors configured to monitor a stream of messages from plural different sources of medical records to identify a new or updated record of the medical records; and
a person matching engine configured to identify one or more suspect records from a system of record (SOR) database, the one or more suspect records identified as being potential matches to the new or updated record that is identified, the person matching engine also configured to determine that the one or more suspect records match the new or updated record, obtain a person entity profile related to the new or updated record from a person entity database, and merge the new or updated record with the one or more suspect records or splitting the new or updated record from the one or more suspect records based on the person entity profile that is obtained.

10. The record matching system of claim 9, wherein the one or more data sync processors are configured to identify the new or updated record in real time.

11. The record matching system of claim 9, wherein the person matching engine is configured to determine that the one or more suspect records match the new or updated record by comparing information contained within the one or more suspect records and the new or updated record using matching rules.

12. The record matching system of claim 11, wherein the person matching engine is configured to be used to author one or more new rules or one or more changes to the matching rules off-line while the matching rules without the one or more new rules or the one or more changes continue to be used to determine whether the one or more suspect records match the new or updated record.

13. The record matching system of claim 12, wherein the person matching engine is configured to test the one or more new rules or the one or more changes to the matching rules off-line using the medical records without merging the new or updated record with the one or more suspect records and without splitting the new or updated record from the one or more suspect records.

14. The record matching system of claim 9, wherein the one or more suspect records include plural suspect records, and the person matching engine is configured to determine that two or more of the suspect records match each other; and further comprising:
one or more update processors configured to merge the two or more of the suspect records with each other.

15. The record matching system of claim 9, wherein the medical records include source identifiers uniquely identifying which of the sources generated the medical records and the person entity database stores the source identifiers associated with each of several different persons,
wherein the person matching engine is configured to merge the new or updated record with the one or more suspect records responsive to the source identifiers in the new or updated record and the one or more suspect records being associated with two or more of the different persons in the person entity database.

16. The record matching system of claim 9, wherein the medical records include source identifiers uniquely identifying which of the sources generated the medical records and the person entity database stores the source identifiers associated with each of several different persons,
wherein the person matching engine is configured to split the new or updated record from the one or more suspect records responsive to the source identifiers in the new or updated record and the one or more suspect records being associated with a single person of the different persons in the person entity database along with one or more other source identifiers.

17. A method comprising:
monitoring a stream of messages from plural different sources of medical records to identify a new or updated record of the medical records;
identifying one or more suspect records from a system of record (SOR) database, the one or more suspect records identified as being potential matches to the new or updated record that is identified;
determining that the one or more suspect records match the new or updated record;
obtaining a person entity profile related to the new or updated record from a person entity database, the person entity profile including source identifiers uniquely identifying which of the sources generated the medical records associated with each of several different persons; and
one or more of (a) merging the new or updated record with the one or more suspect records or (b) splitting the new or updated record from the one or more suspect records,
wherein merging the new or updated record with the one or more suspect records occurs responsive to the source identifiers in the new or updated record and the one or more suspect records being associated with two or more of the different persons in the person entity database, and
wherein splitting the new or updated record from the one or more suspect records occurs responsive to the source identifiers in the new or updated record and the one or more suspect records being associated with a single person of the different persons in the person entity database along with one or more other source identifiers.

18. The method of claim 17, wherein the new or updated record is identified in real time.

19. The method of claim 17, wherein the one or more suspect records are determined to match the new or updated record by comparing information contained within the one or more suspect records and the new or updated record using matching rules, and further comprising:
authoring one or more new rules or one or more changes to the matching rules off-line while the matching rules without the one or more new rules or the one or more changes continue to be used to determine whether the one or more suspect records match the new or updated record.

20. The method of claim 19, further comprising:
testing the one or more new rules or the one or more changes to the matching rules off-line using the medical records without merging the new or updated record with the one or more suspect records and without splitting the new or updated record from the one or more suspect records.
